(19) United States
(12) Patent Application Publication
(10) Pub. No.: US 2026/0138962 A1
(43) Pub. Date: May 21, 2026

(54) SYNTHESIS OF DERIVATIVES OF SIPHONOCHILONE AND THERAPEUTIC USE THEREOF

(71) Applicant: University of Pretoria, Pretoria (ZA)

(72) Inventors: Luke INVERNIZZI, Pretoria (ZA); Phanankosi MOYO, Pretoria (ZA); Vinesh MAHARAJ, Pretoria (ZA)

(21) Appl. No.: 19/117,479

(22) PCT Filed: Oct. 13, 2023
(86) PCT No.: PCT/IB2023/060335

§ 371 Date: Apr. 1, 2025

(30) Foreign Application Priority Data
Oct. 13, 2022 (ZA) 2022/11187

(51) Int. Cl. C07D 307/92; A61K 31/343; A61P 31/14; A61P 31/16

(52) U.S. Cl. CPC C07D 307/92; A61K 31/343; A61P 31/14; A61P 31/16

(57) ABSTRACT

THIS invention relates to the synthesis of derivatives of siphonochilone and therapeutic use thereof in the treatment and CN prevention of respiratory virus disease, in particular influenza or a coronavirus disease.

### BACKGROUND TO THE INVENTION

THIS invention relates to a synthesis of derivatives of siphonochilone and therapeutic use thereof.

The respiratory infectious Covid19 is caused by the novel SARS-CoV-2. The SARS-CoV-2 virus is capable of infecting numerous human cells and systems, most notably those in the upper and lower respiratory tract. The life cycle of the SARS-CoV-2 with the host consists of the following steps: (i) attachment to the host angiotensin-converting enzyme 2 (ACE2) by interaction through the viral spike protein, (ii) penetration into cells through endocytosis, (iii) biosynthesis of viral proteins and (iv) maturation, i.e., making new viral particles ready for release and infection of new cells. Infection of type 2 alveolar epithelial cells in the lungs by SARS-CoV-2 induces a local pulmonary inflammatory response with the resulting cytokine storm, combined with viral replication, consequently causing injury and loss of type 1 and 2 pneumocytes culminating in acute respiratory distress syndrome and extrapulmonary organ failure.

Management strategies employed to curb Covid19 include those of non-pharmaceutical and pharmaceutical nature. Non-pharmaceutical interventions involve social (physical) distancing enforced through ‘lockdown’ measures. Despite their success, these measures have had major negative economic ramifications. Against this background, pharmaceutical interventions have generated a lot of interest. Currently, the most extensively utilized pharmaceutical tool is the Covid19 vaccines. Despite the commendable introduction of these anti-SARS-CoV-2 vaccines, the emergence of new variants is already undermining their efficacy. This has been noted with the Beta-variant against the Oxford-AstraZeneca Covid19 vaccine and other candidates, including ChAdOx1 nCoV-19 and NVX-CoV2373 Covid19, have already been shown to be less effective against Covid19 caused by this variant.

In the absence of widely available anti-viral agents, the discovery of anti-SARS-CoV-2 small molecules represent a smart complementary strategy to treat and prevent Covid19. Currently, there is only one FDA-approved drug for Covid19 treatment, remdesivir. Remdesivir is a broad-spectrum anti-viral agent, which targets the RNA-dependent RNA polymerase enzyme critical for SARS-CoV-2 replication. Remdesivir is only administered to severely ill patients, with only modest efficacy being observed on these patients, despite the fact it attracts a huge price tag. Consequently, there remains an urgent need to discover and develop a cheaper, more efficacious treatment regimen (which ideally can be self-administered, be it either by oral ingestion or inhalation).

The influenza virus has been around since Hippocrates, 2 400 years ago, with numerous outbreaks throughout history.^{[1]} It is a serious global health challenge causing 1 billion cases, resulting in 290 000-650 000 fatalities annually. It is a respiratory infection that is most common amongst old people (>65 years), children (<2 years) and pregnant women and if not treated on time, it may cause bacterial pneumonia, sinus infections and cardiovascular diseases, eventually leading to death. It is important for these patients to get treated/vaccinated annually to prevent the risk of infection.

There are four types of influenza viruses; three known to affect humans (Influenza A, B and C) and only one known to affect animals (Influenza D). Influenza A and B are members of the Orthomyxoviridae family and is responsible for the seasonal spread of flu amongst humans.

Type A is the most common of the four and is an enveloped single negative-strand RNA virus and can only survive by reproducing in a living cell and infecting it. It causes an infectious disease, better known as flu, resulting in high fever, sore throat, headaches, cough, sneezing, tiredness, and muscle pain.

Many influenza viruses are seasonal with new strains surfacing annually in the winter time and can spread easily through coughing and sneezing in a crowded place. The identification of these new strains is important in order for it to be included into the production of vaccines for the upcoming influenza season.^{[14]} With the growing increase in influenza viruses, it is important to try and prevent an epidemic through any means necessary.

It is important to prevent infection since these viruses form an immunity towards vaccines as they mutate. Prevention is most effective in the form of vaccination and has been used for more than 60 years. This proves as protection against infection of circulating viruses and it is important for high-risk patients to get vaccinated annually. It strengthens the immune system and protects the human body usually against three different seasonal viruses.

To prevent spreading of the virus the patient must stay home, get plenty of rest and fluids, and take a drug to relieve the fever and sinus pains (i.e. paracetamol). Antibiotics are ineffective since they are only useful against bacterial infections, and failure to treat the virus can result in high replication efficiency, leading to organ failure or pneumonia, and ultimately death of the patient.

Antiviral drugs can also be used since they cover a wider range of the viruses, whereas vaccines are only effective against three or four viruses.^{[17]} In the case of a pandemic outbreak, various measures can be made to prevent spread such as wearing a facemask and gloves and closing of public places. This might not be effective and a vaccine may take too long to be developed and approved, therefore presenting antiviral drug as a first line of defense.

It is accordingly an object of the invention to provide a synthesis of the derivatives of siphonochilone and therapeutic use thereof, for treatment of respiratory virus disease, in particular for treating coronavirus disease (SARS-CoV-2) and influenza or preventing or reducing transmission of coronavirus disease (SARS-CoV-2) and influenza, that will at the least, partially alleviate the above problems.

### SUMMARY OF THE INVENTION

According to the invention there is a process provided for preparing (4aR,5S,8aS,9aR)-9a-hydroxy-3,5,8a-trimethyl-4a,5,9,9a-tetrahydronaphtho[2,3-b]furan-2,8(4H,8aH)-dione and/or derivatives thereof, comprising of the following steps:
(a) dissolving (4aS,5R,8aR)-3,5,8a-Trimethyl-4a,5,8a,9-tetrahydronaphtho[2,3-b]furan-8(4H)-one (siphonochilone) in a polar protic/aprotic solvent such as water, methanol, ethanol, acetonitrile, isopropanol, Dimethylsulfoxide (DMSO), Tetrahydrofuran (THF) or acetone and preferably acetone,
(b) adding an oxidizing agent such as methylthioninium chloride (methylene blue) to step (a) to form a mixture,
(c) agitating the mixture obtained in step (b) in the presence of UV light, sunlight, or infrared light and optionally bubbling in oxygen and typically for 2 to 3 days; thereby
producing (4aR,5S,8aS,9aR)-9a-hydroxy-3,5,8a-trimethyl-4a,5,9,9a-tetrahydronaphtho[2,3-b]furan-2,8(4H,8aH)-dione, and/or derivatives thereof.

At step b) methylthioninium chloride may be added to the mixture at ratios of 200:1 (m/m) (and upwards, up to 1:1 1 (m/m)) mixture to methylthioninium chloride.

The product/s from step c) may be purified using chromatographic techniques such as high-performance liquid chromatography (HPLC).

The product may be purified further by dissolving the compound from c) in a solvent such as methanol or ethanol and crystallized.

According to another embodiment of the invention, there is provided a lactone of siphonochilone compound selected from:

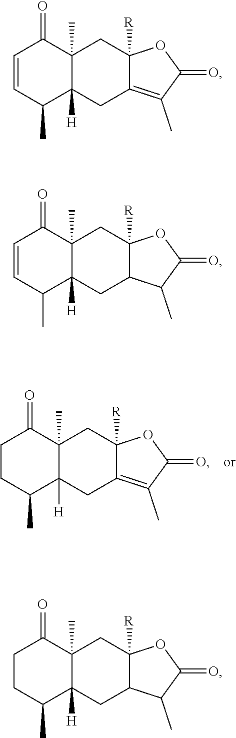

wherein R may be OCH₃, NH₂, CH₂OCH₃, C(O)CH₃ or CH₂OH.

The invention also relates to a pharmaceutical formulation comprising a compound described above.

The invention also relates to a method for treating a respiratory virus disease, the method comprising of the administration of a therapeutically effective amount of one (4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof.

The invention further relates to (4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof, for use in a method for treating a respiratory virus disease, wherein the method comprises of administering a therapeutically effective amount of one or more of said compounds or composition to a subject.

The invention further relates to a method for preventing or reducing the transmission of a respiratory virus disease, the method comprising the administration of a therapeutically effective amount of (4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof to a subject in need thereof.

The invention further relates to (4aR,5S,8aS,9aR)-9a-hydroxy-3,5,8a-trimethyl-4a,5,9,9a-tetrahydronaphtho[2,3-b]furan-2,8(4H,8aH)-dione or a derivate thereof, for use in a method for preventing or reducing the transmission of a respiratory virus disease, the method comprises administering a therapeutically effective amount of one or more of said compounds to a subject in need thereof.

The respiratory virus disease is the influenza virus, respiratory syncytial virus, parainfluenza viruses, metapneumovirus, rhinovirus, coronaviruses, adenoviruses, and bocaviruses, typically an influenza or a coronavirus disease.

The coronavirus disease may be COVID-19, SARS-CoV-2 or variants thereof, such as the Alpha (B.1.1.7), Beta (B.1.351), Gamma (P.1) or Delta (B.1.617.2) variants.

The influenza virus may be a Influenza A-type virus.

The compound or composition may be administered orally, by inhalation, intranasal, intravenous injection, subcutaneous injections, intramuscular, per rectal or sublingual.

The subject may be a human.

### BRIEF DESCRIPTION OF THE DRAWINGS

FIG. 1 is a chemical reaction overview of a semi-synthetic conversion of siphonochilone to the hydroxylated lactone of siphonochilone (HLS) produced in the present invention;

FIG. 2 shows the chemical structures of derivatives of the hydroxylated lactone of siphonochilone (HLS);

FIG. 3 is a single crystal X-ray diffraction of hydroxylated lactone of siphonochilone (HLS) produced;

FIG. 4 is a graph showing the activity of HLS against SARS-CoV-2 isolates in vitro by observing a reduction of the cytopathic effect (CPE) of the virus;

FIG. 5 is a graph showing the activity of HLS against Influenza A isolates in vitro by observing a reduction of the cytopathic effect (CPE) of the virus;

FIG. 6 is a dose-dependent graph showing the activity of HLS against Influenza A isolates in vitro by observing a reduction of the cytopathic effect (CPE) of the virus; and

FIG. 7 is a dose-dependent graph showing the activity of ribavirin (positive control) against Influenza A isolates in vitro by observing a reduction of the cytopathic effect (CPE) of the virus.

### DESCRIPTION OF EMBODIMENTS OF THE INVENTION

The present invention relates to the technical field of organic synthesis and therapeutic use of a formulation or medicament containing the hydroxylated form of siphonochilone for the treatment of a respiratory virus disease, such as the influenza virus, respiratory syncytial virus, parainfluenza viruses, metapneumovirus, rhinovirus, coronaviruses, adenoviruses, and bocaviruses, typically an influenza or a Covid19 by inhibiting replication of the novel virus (SARS-CoV-2).

The invention provides for (i) the novel stereo-selective semi-synthetic conversion of siphonochilone using acetone and methylene blue to produce the hydroxylated lactone of siphonochilone and (ii) a therapeutic formulation or medicament containing the hydroxylated lactone of siphonochilone as a therapeutic agent for administration, either by oral ingestion (for example tablet, capsule, powder or granules) or inhalation (for example solutions, suspensions or powders), intranasal, intravenous injection, subcutaneous injections, intramuscular, per rectal or sublingual to either prevent or reduce transmission of influenza or coronavirus disease, in particular, the novel severe acute respiratory syndrome coronavirus 2 (SARS-CoV-2) or to treat coronavirus disease, in particular 2019 (Covid19) caused by SARS-CoV-2 in humans as well as any variants thereof.

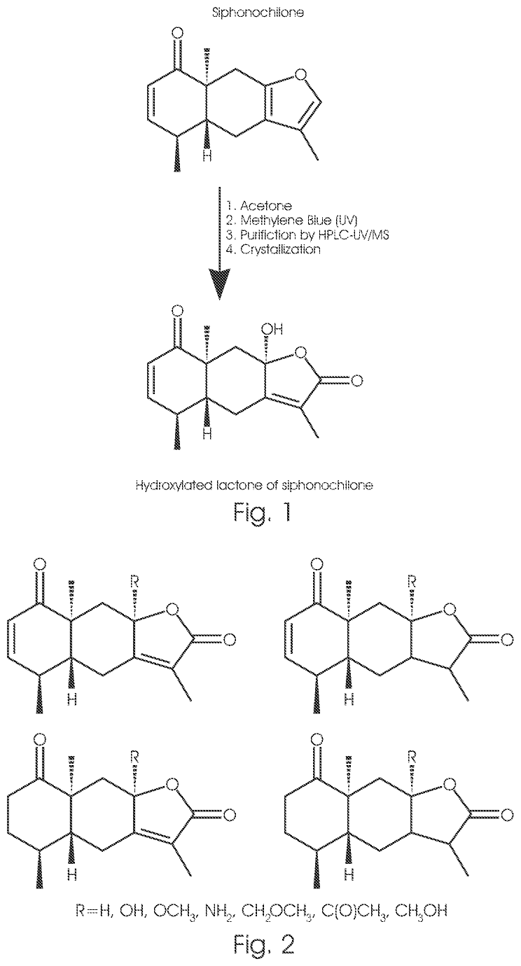

The invention relates to:
(i) the novel stereo-selective procedure for the synthetic conversion of siphonochilone ((4aS,5R,8aR)-3,5,8a-Trimethyl-4a,5,8a,9-tetrahydronaphtho[2,3-b]furan-8(4H)-one) (shown in FIG. 1 [1]) using acetone and methylene blue to produce the hydroxylated version of siphonochilone herewith “HLS”, where HLS is (4aR,5S,8aS,9aR)-9a-hydroxy-3,5,8a-trimethyl-4a,5,9,9a-tetrahydronaphtho[2,3-b]furan-2,8(4H,8aH)-dione (shown in FIG. 1 [2]) and/or its derivatives thereof.
(ii) use of a therapeutic formulation or medicament containing the HLS and/or one or more of its derivatives (R═H, OH, OCH₃, NH₂, CH₂OCH₃, C(O)CH₃, CH₂OH) (shown in FIG. 2) for administration, either by oral ingestion or inhalation, intranasal, intravenous injection, subcutaneous injections, intramuscular, per rectal or sublingual to either reduce or prevent transmission of SARS-CoV-2 or to treat Covid19 patients. The compounds of interest demonstrate activity against the Alpha (B.1.1.7), Beta (B.1.351), Gamma (P.1) and Delta (B.1.617.2) variants of the virus.

The chemical conversion involves converting siphonochilone, a naturally occurring furano sesquiterpenoid, via an oxidation reaction (photooxidation) into the hydroxylated form, ((4aR,5S,8aS,9aR)-9a-hydroxy-3,5,8a-trimethyl-4a,5,9,9a-tetrahydronaphtho[2,3-b]furan-2,8(4H,8aH)-dione), by synthetic means.

### EXAMPLES

### Example 1

The process involves dissolving siphonochilone in acetone before the addition of methylene blue in a 200:1 (and upwards) ratio (m/m). The mixture is agitated in the presence of UV light (or sunlight) for 2-3 days.

The reaction results in the production of ((4aR,5S,8aS,9aR)-9a-hydroxy-3,5,8a-trimethyl-4a,5,9,9a-tetrahydronaphtho[2,3-b]furan-2,8(4H,8aH)-dione) as well as its derivatives.

To purify the compounds of interest, the mixture is dried and purified using HPLC-UV/MS on a C18 reverse phase column. The purified compound is dissolved in deuterated methanol (MeOD) or deuterated dichloromethane (CD₂Cl₂) for NMR analysis.

The purified sample is further dissolved in methanol before being allowed to crystalise by slow evaporation.

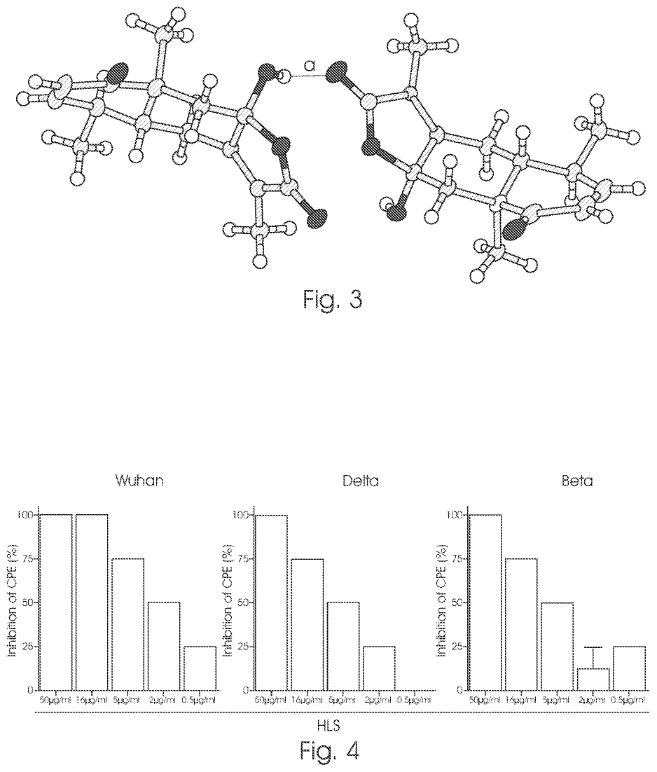

The compound identity is confirmed with ¹H and ¹³C NMR (Table 1) and the absolute configuration by single crystal X-ray crystallography using a diffractometer (SCXRD) (FIG. 3). Table 1 describes the assignments of the numbered H (hydrogen) or C (carbon) shown in the Figure with their corresponding signals in the ¹H and ¹³C NMR spectra. Assignments are reported as chemical shifts in ppm, relative from the reference standard TMS and their ¹H-¹H coupling constants (J values) based on their splitting patterns (singlet—s, doublet—d, triplet—t, multiplet—m). Data was obtained using a 400 MHz Bruker NMR instrument.

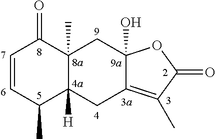 [SMILES: CC1=C2C[C@@H]3[C@@H](C)C=CC(=O)[C@@]3(C)C[C@@]2(O)OC1=O]

To further confirm the absolute configuration of the compound, the single crystals were analysed on a Rigaku XRD instrument where their packing and unit cells with their accompanying atomic configuration was confirmed.

The conversion is found to be stereoselective and, after purification, yields a compound with >95% purity by HPLC-UV and NMR.

TABLE 1
¹H and ¹³C of the HLS in CD₂Cl₂

| Position | ¹H (ppm) | ¹³C (ppm) |
|---|---|---|
| 2 | | 172.33 |
| 3 | | 123.0 |
| 3a | | 158.97 |
| 4ₐₓ | 2.38 (m) | 24.61 |
| 4_{eq} | 2.84 (dd, J = 13.63, 3.73) | 24.61 |
| 4a | 1.56 (m) | 50.46 |
| 5 | 2.48 (m) | 34.44 |
| 6 | 6.67 (dd, J = 10.07, 1.94) | 154.36 |
| 7 | 5.83 (dd, J = 10.09, 2.77) | 126.3 |
| 8 | | 203.5 |
| 8a | | 45.38 |
| 9_{eq} | 1.63 (d, J = 14.21) | 44.18 |
| 9ₐₓ | 2.59 (d, J = 14.41) | 44.18 |
| 9a | | 103.9 |
| 3-Me | 1.79 (d, J = 1.31) | 8.56 |
| 5-Me | 1.22 (d, J = 7.16) | 18.49 |
| 8a-Me | 1.33 (s) | 17.12 |
| 9a-OH | 3.84 (s) | |

### Example 2

Anti-viral Bioassays against SARS-CoV-2: The appropriate volume of Vero E6 cells are prepared (˜1×10⁶ cells per 96 well plate). The cells are allowed to adhere for 2-4 h in an incubator. Sufficient dilutions of the compound are aliquoted into the wells and allowed to incubate with the Vero E6 cells for 2-4 hours. Thereafter, in a BSL3 facility, the Vero E6 cells containing the HLS are infected with the diluted SARS-CoV-2 virus and the various variants of interest. Each dilution is plated in duplicates for increased accuracy. The cells containing the compound and virus are incubated at 37° C. for 2 days with regular observation for cytopathic effect (CPE) and disturbance of the confluent monolayer in the negative control. After 2 days, the supernatant is removed, and a 3.7% dilution of formaldehyde is added to fix the cells. The mixture is given 1 h to allow for adequate fixation. To each well, 0.5% Crystal Violet is added and incubated with the cells for 5-10 min. Thereafter the cells are washed with tap water and allowed to dry before any observation is carried out.

HLS is potent against the wild type of the Wuhan isolate and variants of it, namely Beta and Delta (shown in Table 2 and FIG. 4). Remdesivir was used as the positive control and exhibited 100% inhibition against all variants at 3.77 μg/ml.

TABLE 2
Antiviral activity of HLS against the various SARS-
CoV-2 variants, given as percentage inhibition.

| | | SARS-CoV- | Concentration (μg/ml) | | | | |
|---|---|---|---|---|---|---|---|
| | Sample | 2 variant | 50 | 16 | 5 | 2 | 0.5 |
| % | HLS | Wuhan | 100 | 100 | 75 | 50 | 25 |
| Inhibition | | Beta | 100 | 75 | 50 | 25 | 25 |
| | | Delta | 100 | 75 | 50 | 25 | 0 |

The following table reports the observed IC₅₀ values of HLS against the SARS-CoV-2 Wuhan, Delta and Beta strain of the virus, in-vitro where IC₅₀ refers to the concentration (μg/ml) of the drug (viz. HLS) required to inhibit 50% of the virus population from replicating and/or viral death. Inhibition in this context refers to the ability of HLS to prevent replication, death or additional growth of the SARS-CoV-2 virus and hence prevent any observable CPE (Cytopathic effect) or damage to the other healthy cells and hence protect them and prevent further infection.

TABLE 3
CPE of HLS against SARS-CoV-2

| | IC₅₀ (μg/ml) | | |
|---|---|---|---|
| Compound | Wuhan | Delta | Beta |
| HLS | 3.03 | 6.15 | 8.93 |

### Example 3

Anti-viral Bioassays against Influenza A: The in vitro screening of selected samples against a strain of the Influenza A-type virus was performed followed by evaluation of their toxicity (FIG. 4.4). A plaque assay was first developed to determine the viral titre as plaque-forming units per mL (pfu/mL) of two strains of Influenza A-type viruses (108 617 and 61332 065) (FIG. 4.4). This was then followed by a Cytopathic Effect Reduction Assay against Influenza A (CERA-1) to determine the inhibition and toxicity of the test samples against the selected strain from the plaque assay.

Two strains of the Influenza A-type virus (108 617 and 61332 065), both available at the Department of Biomedicine at the University of Basel, Switzerland, were used for the plaque assay to determine which has the highest titre and is most suitable for the Cytopathic Effect Reduction Assay against Influenza A (CERA-1). Well numbers 1-12 were infected with strain 108 617 and well numbers 13-24 with strain 61332 065. A viral dilution of each strain was prepared with 10 μL of the virus stock and 990 μL DMEM, starting with a 1:100 dilution and followed by a 1:5 serial dilution in 500 μL DMEM (without FBS). The plate was left to incubate at 37° C. with 5% CO₂ for 4 days.

Growth and cytopathic effect (CPE) formation was observed regularly. Cell confluency as monolayer in the negative control wells and clear CPE with plaque formation (30-60%) in the infected wells were verified under the microscope.

After 4 days, the agarose was removed by turning the plate over a liquid waste container with pre-added 1% Korsolex for virus deactivation. The cells were fixed by adding 250 μL of 3.60% formaldehyde (1:10 dilution from 37% in PBS) to each well and incubated for 45 min at room temperature. The formaldehyde was removed and the cells were stained with 0.50% crystal violet and incubated for 5 min at room temperature. Excess dye was removed and washed with distilled H₂O and the blue stained plaques were counted. The titre of a virus stock was reported as plaque-forming units (PFU) per mL:
MDCK cells were seeded in a T75 flask and incubated with 5% CO₂ at 37° C. for 48 hours before infection. A Neubauer chamber was used to count the seeds: 3.00×10⁶ cells in 10 mL for one 96-wp. To have enough cells for two well plates to perform the inhibition and cytotoxicity studies, the MDCK cells were resuspended in 10 mL and 6.0×10⁶ cells were available.

All cells were confluent (100%) and 7.90 mL were suspended with DMEM (2.10 mL) to make up 10 mL per well plate. The cells were thoroughly washed once with PBS and 100 μL were suspended to each well (3.00×10⁴ cells/well) of a 96-wp. The infectious media (DMEM-T) was prepared for all plates by adding 49 mL of DMEM (without serum) with 1.00 mL of 0.10% TPCK treated trypsin (final concentration of 1.00 μg/mL) and dispensing 100 μL of media to each well of the 96-wp. Influenza A-type virus strain 108 617 was selected and used for the virus dilution. The TCID₅₀ of the virus was determined during the plaque assay and 10 mL of virus stock was prepared per 96-wp, MOI 0.001. Each well was infected with 190 μL of the viral stock, except for six wells that represented the cell—and solvent controls.

Hydroxylated lactone (HLS) were resuspended in 0.5% DMSO before dilution. Twelve dilutions were made, starting at 50, 40 and 30 μg/mL, and were prepared as 10-fold concentrations with a 3-step serial dilution of 1:2. For each plate 1.00 mL of each dilution as prepared and 10 μL was dispensed to each well. HLS dilution was done in triplicate to produce reproducibility. A cell titre blue protocol was used for the readout of viral growth and 20 μL of titre blue was used to stain the cells. The plates were incubated for 1 h in the BSL-2 incubator and the solution's absorbance in each well was measured using a TecanSaphire II spectrometer with excitation at 545 nm and emission at 590 nm, both with a bandwidth of 20 nm. The plates were again measured after 4-6 h. The reaction was stopped and stabilized with 3% SDS and again measured 24 h later.

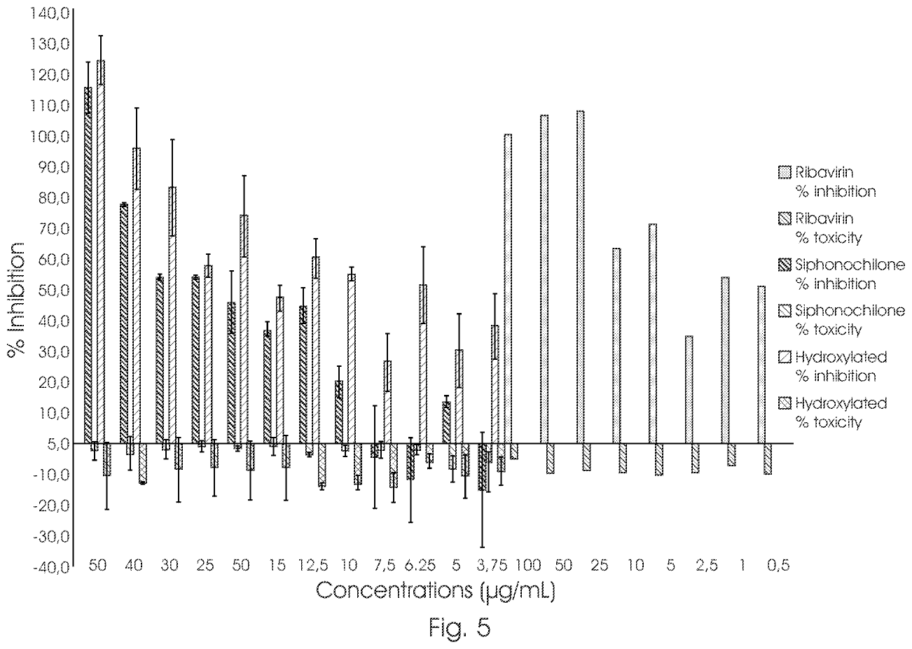

FIG. 5 shows the antiviral efficacy of HLS in MDCK cells infected with Influenza A-type virus (strain 108 617). Treatment was at different concentrations with 3 steps of serial dilution of 1:2 at a dose-response manner from 50.0 to 3.75 μg/mL.

HLS (FIG. 5—purple bars) exhibited excellent antiviral activity at all test concentrations with >100% and >30% inhibition at 50.0 μg/mL and 3.75 μg/mL, respectively, with no toxicity. At 50 μg/mL the lactone showed >123% inhibition which compared well with ribavirin, >105% inhibition, at the equivalent test concentration.

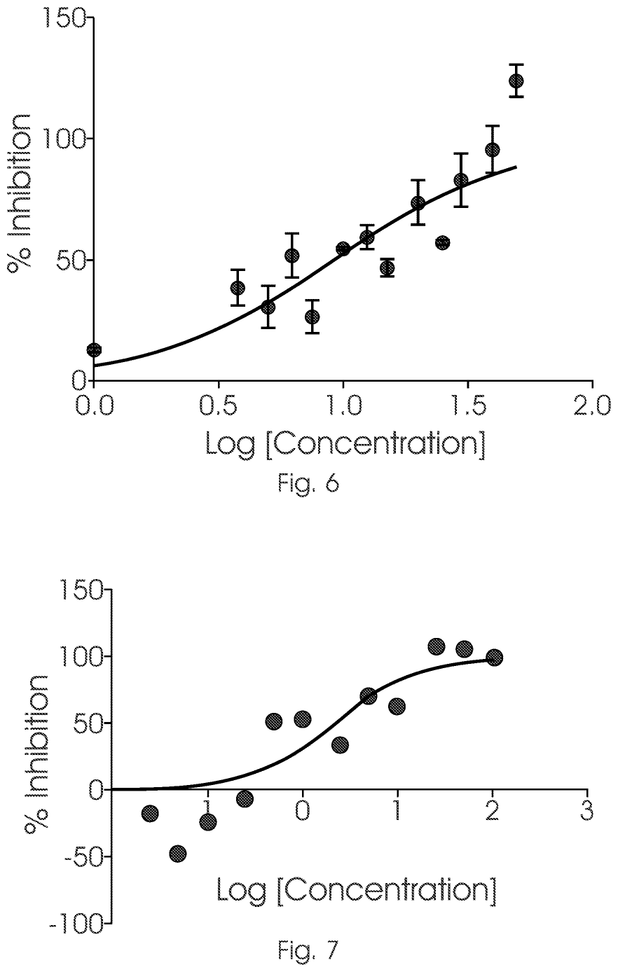

HLS reported significantly good activity against the Influenza A-type virus and showed good antiviral activity compared to the ribavirin. As with the HLS, and ribavirin, there was a gradual decrease in the inhibition as the test concentrations decreased. Using the test concentrations and percentage inhibition of each of these compounds and ribavirin, the IC₅₀ was calculated using GraphPad Prism. This resulted in the IC₅₀ results of the hydroxylated lactone (HLS) as 9.20 μg/mL (R²=0.71) (FIG. 6) and ribavirin as 2.16 μg/mL (R²=0.79) (FIG. 7).

1. A process provided for preparing (4aR,5S,8aS,9aR)-9a-hydroxy-3,5,8a-trimethyl-4a,5,9,9a-tetrahydronaphtho[2,3-b]furan-2,8(4H,8aH)-dione and/or derivatives thereof, comprising of the following steps:
(a) dissolving (4aS,5R,8aR)-3,5,8a-Trimethyl-4a,5,8a,9-tetrahydronaphtho[2,3-b]furan-8(4H)-one in a polar protic/aprotic solvent,
(b) adding an oxidizing agent to step (a) to form a mixture, and
(c) agitating the mixture obtained in step (b) in the presence of UV light, sunlight, or infrared light;
thereby
producing (4aR,5S,8aS,9aR)-9a-hydroxy-3,5,8a-trimethyl-4a,5,9,9a-tetrahydronaphtho[2,3-b]furan-2,8(4H,8aH)-dione, and/or derivatives thereof.

2. The process claimed in claim 1, wherein the polar protic/aprotic solvent at Step (a) is selected from water, methanol, ethanol, acetonitrile, isopropanol, Dimethylsulfoxide (DMSO), Tetrahydrofuran (THF), and acetone.

3. The process claimed in claim 2, wherein the polar protic/aprotic solvent is acetone.

4. The process claimed in claim 1, wherein the oxidizing agent at step (b) is methylthioninium chloride.

5. The process claimed in claim 4, wherein at step b) methylthioninium chloride is added to the mixture at ratios of 200:1 (m/m) and upwards, mixture to methylthioninium chloride.

6. The process claimed in claim 1, wherein at step (c) oxygen is bubbled through the mixture.

7. The process claimed in claim 1, wherein step (c) is carried out for 2 to 3 days.

8. The process claimed in claim 1, wherein the product/s from step c) are purified using a chromatographic technique.

9. The process claimed in claim 8, wherein the chromatographic technique is high-performance liquid chromatography (HPLC).

10. The process claimed in claim 8, wherein the product is purified further by dissolving the compound from step (c) in a solvent such as methanol or ethanol and crystallized.

11. The process claimed in claim 1, wherein the derivative of (4aR,5S,8aS,9aR)-9a-hydroxy-3,5,8a-trimethyl-4a,5,9,9a-tetrahydronaphtho[2,3-b]furan-2,8(4H,8aH)-dione is a lactone selected from: 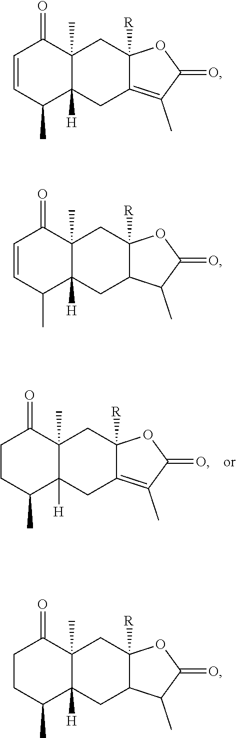
wherein R is OCH₃, NH₂, CH₂OCH₃, C(O)CH₃ or CH₂OH.

12. A compound which is a lactone derivative of (4aR,5S,8aS,9aR)-9a-hydroxy-3,5,8a-trimethyl-4a,5,9,9a-tetrahydronaphtho[2,3-b]furan-2,8(4H,8aH)-dione selected from: 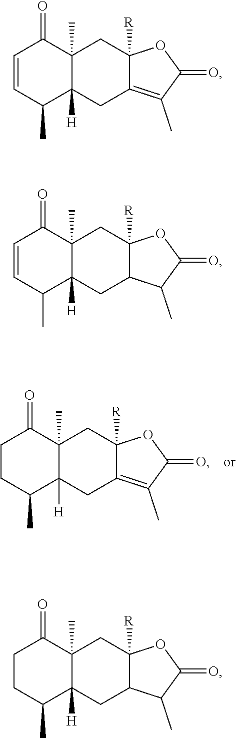
wherein R is OCH₃, NH₂, CH₂OCH₃, C(O)CH₃ or CH₂OH.

13. A pharmaceutical formulation comprising a compound as claimed in claim 12.

14. A method for treating a respiratory virus disease, the method comprising of the administration of a therapeutically effective amount of ene-(4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof.

15. A compound (4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof, for use in a method for treating a respiratory virus disease, the method comprising administering a therapeutically effective amount of the compound or derivative to a subject in need thereof.

16. A method for preventing or reducing the transmission of a respiratory virus disease, the method comprising administrating a therapeutically effective amount of (4aR,5S,8aS,9aR)-9a-hydroxy-3,5,8a-trimethyl-4a,5,9,9a-tetrahydronaphtho[2,3-b]furan-2,8(4H,8aH)-dione or a derivative thereof to a subject in need thereof.

17. A compound (4aR,5S,8aS,9aR)-9a-hydroxy-3,5,8a-trimethyl-4a,5,9,9a-tetrahydronaphtho[2,3-b]furan-2,8(4H,8aH)-dione or a derivate thereof, for use in a method for preventing or reducing the transmission of a respiratory virus disease, the method comprising administering a therapeutically effective amount of the compound or derivative to a subject in need thereof.

18. The method claimed in claim 14 or claim 16, or the compound or derivative for use claimed in claim 15 or claim 16, wherein the respiratory virus disease is the influenza virus, respiratory syncytial virus, parainfluenza viruses, metapneumovirus, rhinovirus, coronaviruses, adenoviruses, and bocaviruses, typically an influenza or a coronavirus disease.

19. The method, or compound or derivative for use claimed in claim 18, wherein the respiratory virus disease is a coronavirus disease.

20. The method, or compound or derivative for use claimed in claim 19, wherein the coronavirus disease is COVID-19, SARS-CoV-2 or variants thereof.

21. The method, or compound or derivative for use claimed in claim 20, wherein the coronavirus disease are the Alpha (B.1.1.7), Beta (B.1.351), Gamma (P.1) or Delta (B.1.617.2) variants.

22. The method, or compound derivative for use claimed in claim 18, wherein the respiratory virus disease is an influenza virus.

23. The method, or compound or derivative for use claimed in claim 22, wherein the influenza virus is an Influenza A-type virus.

24. The method, or compound or derivative for use claimed in claim 18, wherein the compound or derivative is administered orally, by inhalation, intranasal, intravenous injection, subcutaneous injections, intramuscular, per rectal or sublingual.

25. The method, or compound or derivative for use claimed in claim 18, wherein the subject is a human.

26. The method, or compound or derivative for use claimed in claim 18, wherein the derivative of (4aR,5S,8aS,9aR)-9a-hydroxy-3,5,8a-trimethyl-4a,5,9,9a-tetrahydronaphtho[2,3-b]furan-2,8(4H,8aH)-dione is a lactone selected from: 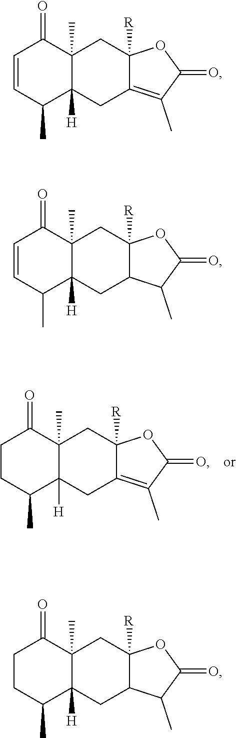
wherein R is OCH₃, NH₂, CH₂OCH₃, C(O)CH₃ or CH₂OH.
